(19) United States
(12) Patent Application Publication
(10) Pub. No.: US 2002/0142047 A1
(43) Pub. Date: Oct. 3, 2002

(54) Microsphere delivery of mucin peptides

(76) Inventors: Mark E. Johnson, Bellevue, WA (US); Tricia Cecil, Bellevue, WA (US); Olivera J. Finn, Pittsburgh, PA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/054,488

(22) Filed: Jan. 22, 2002

Related U.S. Application Data
(60) Provisional application No. 60/262,699, filed on Jan. 19, 2001.

(51) Int. Cl. A61K 9/16; A61K 38/10

(52) U.S. Cl. 424/491; 514/13; 514/62

(57) ABSTRACT

A mucin peptide, such as MUC-1, encapsulated in a biodegradable polymeric microsphere is disclosed. The encapsulated mucin peptide breaks tolerance of helper T cells as it elicits a stronger immune response and provides improved protection against tumor challenge than direct administration of peptide, alone or with an adjuvant. The encapsulated mucin peptide can be used in a vaccine composition, and can be used in a method for delivering a mucin peptide to a subject, as well as in a method treating or preventing a cancer associated with reduced glycosylation of MUC-1.

This application claims the benefit of priority to U.S. provisional patent application No. 60/262,699, filed Jan. 19, 2001, the entire contents of which are incorporated herein by reference.

Throughout this application various publications are referenced. The disclosures of these publications in their entireties are hereby incorporated by reference into this application in order to describe more fully the state of the art to which this invention pertains.

### TECHNICAL FIELD OF THE INVENTION

The invention relates to formulations, compositions and methods that can be used for the delivery of vaccines comprising mucin peptides, such as MUC-1, and use of such vaccines for the treatment and prevention of cancer. More particularly, the invention relates to microspheres and adjuvants for more efficient and effective delivery of mucin vaccines.

### BACKGROUND OF THE INVENTION

Immune cells that have shown autologous tumor reactivity have been isolated from patients with a variety of tumor types and this is clear evidence that at least some human tumors can elicit a cellular immune response. Lymphocytes with immune reactivity have been isolated from tumors and draining lymph nodes, and these tumor infiltrating lymphocytes (TIL) have been used in adoptive immune transfer protocols with some success, especially in patients with melanoma. The antigens responsible for this tumor-specific immune reactivity remain elusive. One family of tumor-associated molecules that can induce a specific immune response is the mucin MUC-1.

MUC-1 mucin is a transmembrane glycoprotein that is present on ductal epithelia of the pancreas, ovary, breast, lung and prostate. In normal tissues, MUC-1 mucin is heavily glycosylated with O-linked carbohydrates. Over 50% of the molecular weight of mucin is contributed by the carbohydrate side chains, which are linked to serine and threonine residues of the polypeptide core. Much of the glycosylation is found within regions of tandemly repeated sequences of 10-81 amino acids per repeat. Mucins are produced by cells of epithelial origin and are abundantly present on the luminal surface of these cells as they form glands. In contrast, in adenocarcinomas of epithelial origin, the degree of glycosylation is markedly reduced with a corresponding loss in luminal polarity. The effect of hypoglycosylation and loss of luminal polarity is to expose the extracellular region of the protein, which consists largely of a tandemly repeating peptide sequence of 20 amino acids. Concurrent with this unmasking, CTLs and antibody responses that are specific for epitopes within the tandem repeat region of MUC-1 are generated in cancer patients. Neither immune response, however, is effective at controlling disease.

In vitro studies in which MUC-1 peptide-loaded dendritic cells (DCs) were used to prime human CD4+ T cells suggest that it is necessary to use high concentrations of peptide and professional antigen presenting cells to activate MUC-1 specific helper T cell responses. In vivo studies have shown that peptide-pulsed DC are able to generate both CD4+ and CD8+ responses in wild-type mice; however, such a DC vaccine is incapable of overcoming CD4+ T cell tolerance in MUC-1 transgenic mice. Thus, there remains a need for new ways to augment immunity to tumor MUC-1 and to develop an effective immunotherapy for adenocarcinomas. As disclosed in further detail herein, this and other related needs are fulfilled by the present invention that provides formulations, compositions and methods employing biodegradable microspheres for the delivery of MUC-1 peptides.

### SUMMARY OF THE INVENTION

The invention provides a composition comprising a mucin peptide and a biodegradable microsphere, typically in the form of a mucin peptide encapsulated in a biodegradable polymeric microsphere. Also provided is an encapsulated nucleic acid encoding a mucin peptide. A preferred mucin peptide is a MUC-1 peptide. Preferred MUC-1 peptides comprise one or more repeats of the peptide sequence GVTSAPDTRPAPGSTAPPAH (SEQ ID NO:1). More preferred are MUC-1 peptides comprising two or more repeats of the peptide sequence GVTSAPDTRPAPGSTAPPAH (SEQ ID NO:1).

The encapsulated mucin peptide or mucin-encoding nucleic acid elicits a stronger immune response and provides surprisingly improved protection against tumor challenge as compared to direct administration of peptide, alone or with an adjuvant. The encapsulated mucin peptide or nucleic acid encoding a mucin peptide can be used in a vaccine composition, and can be used in a method for delivering a mucin peptide to a subject, as well as in a method of stimulating an immune response to MUC-1 in a subject, a method of inhibiting tumor growth in a subject having a cancer associated with reduced glycosylation of MUC-1, and in a method of prolonging survival in a subject having a cancer associated with reduced glycosylation of MUC-1, as well as in methods for treating or preventing a cancer associated with reduced glycosylation of MUC-1.

In one embodiment, at least about 90% of the microspheres are about 1 to about 20 μm in diameter, preferably about 3 to about 10 μm, and more preferably about 6 to about 8 μm in diameter. Microspheres in this size range are well-suited to be phagocytosed by antigen-presenting cells, leading to effective T cell stimulation.

The microspheres of the invention preferably comprise a biodegradable polymer, such as poly(lacto-co-glycolide) (PLG), poly(lactide), poly(caprolactone), poly(hydroxybutyrate) and/or copolymers thereof. Exemplary microspheres suitable for use in the formulations, compositions and methods of the present invention are disclosed in U.S. patent application No. 09/901,829, incorporated herein by reference in its entirety. Alternatively, the microspheres can comprise another wall-forming material. These materials may be used alone, as physical mixtures (blends), or as copolymers. The delivery system can further comprise an adjuvant, preferably an aminoalkyl glucosaminide 4-phosphate (AGP), 2-deoxy-2-amino-beta-D-glucopyranose (glucosamine) glycosidically linked to a cyclic aminoalkyl (aglycon) group (cyclic AGP) or MPL. Alternatively, or in addition, the delivery system can further comprise a saponin, preferably QuilA, QS-21 or GPI-100.

The invention further provides a method for encapsulating mucin peptides or mucin-encoding nucleic acids in microspheres. The method comprises dissolving a polymer in a solvent to form a polymer solution; adding an aqueous solution containing nucleic acid molecules to the polymer solution to form a primary emulsion; homogenizing the primary emulsion; mixing the primary emulsion with a process medium comprising a stabilizer to form a secondary emulsion; and extracting the solvent from the secondary emulsion to form microspheres encapsulating nucleic acid molecules. The method can further comprise subsequent steps of washing, freezing and lyophilizing the microspheres.

In a preferred embodiment, the polymer comprises PLG. In some embodiments, the PLG can include ester end groups or carboxylic acid end groups, and have a molecular weight of from about 4 kDa to about 120 kDa, or preferably, about 8 kDa to about 65 kDa. The solvent can comprise, for example, dichloromethane, chloroform, or ethylacetate. In some embodiments, the polymer solution further comprises a cationic lipid and/or an adjuvant, such as MPL. Examples of stabilizers include, but are not limited to, carboxymethylcellulose (CMC), polyvinyl alcohol (PVA), polyvinyl pyrrolidone (PVP), or a mixture thereof. The stabilizer can optionally further comprise a cationic lipid. In some embodiments, the stabilizer comprises from about 0 to about 10% of the process medium, or preferably, about 1% to about 5% of the process medium. In some embodiments, the solvent comprises an internal water volume of from about 0.001% to about 0.5%; and/or the aqueous solution comprises an ethanol content of from about 0% to about 75% (v/v).

The nucleic acid molecule preferably comprises DNA. In one embodiment, the aqueous solution comprises about 0.2 to about 12 mg/ml DNA. The aqueous solution can optionally further comprise a stabilizer, such as BSA, HSA, or a sugar, or an adjuvant, such as the saponin compounds QuilA, QS-21 and GPI-100. Exemplary saponins suitable for use in the formulations, compositions and methods of the present invention are disclosed in U.S. Pat. Nos. 6,262,029, 6,080,725, 5,977,081 and 5,583,112, each of which is incorporated herein by reference. In one embodiment, the DNA comprises a plasmid of about 2 kb to about 12 kb, preferably, about 3 kb to about 9 kb.

The invention additionally provides a composition comprising mucin peptides or nucleic acid molecules encapsulated in microspheres produced by a method of the invention. Preferably, the composition further comprises an adjuvant and/or a saponin.

### BRIEF DESCRIPTION OF THE FIGURES

FIG. 1 is a bar graph showing interferon gamma (IFN-γ) production in MUC-1 transgenic mice treated with MUC-1 peptide, as measured by number of IFN- spots per 10⁵ cells in peptide-pulsed dendritic cell group (solid and white bars), AS2+ peptide group (diagonally striped bars), GM-CSF+ peptide group (striped bars), and control group (stippled bars); + indicates antigen-positive and − indicates antigen-negative mice.

FIG. 2 is a bar graph showing interferon gamma (IFN-γ) production in mice treated with MUC-1 peptide, as measured by percentage of CD3 cells positive for IFN-γ in peptide-pulsed dendritic cell group (solid and white bars), AS2+ peptide group (diagonally striped bars), GM-CSF+ peptide group (striped bars), and control group (stippled bars); + indicates antigen-positive and − indicates antigen-negative mice.

FIGS. 3A and 3B are survival plots showing tumor rejection, plotted as percent surviving at the indicated number of days following tumor challenge for wild type mice (FIG. 3A) and MUC-1 transgenic (FIG. 3B) mice. Groups were treated as follows: dendritic cells pulsed with MUC-1 peptide (squares), GM-CSF+ peptide (diamonds), AS2+ peptide (circles), and control (triangles).

FIGS. 4A and 4B are graphs depicting IgM (FIG. 4A) and IgG (FIG. 4B) responses of mice immunized with MUC-1 peptide-loaded microspheres. Mice were immunized three times three weeks apart. Ten days following the last boost, the mice were bled for serum. MUC-1 specific ELISA were carried out as described in Example 6.

FIGS. 5A-D are bar graphs depicting cytokine (IFN-γ) production by MUC-1 specific T cells from immunized MUC-1 transgenic ELISPOT assays carried out using lymph node (LN) cells from immunized (FIGS. 5A-B) and control (FIGS. 5C-D) mice stimulated for 40 hours with 40-mer MUC-1 peptide-pulsed (FIGS. 5A, 5C) or no peptide control (FIGS. 5B, 5D) DC. The lymph node cells were pooled from four mice per group. Anti-CD4 or anti-CDS antibodies were added to the T cells prior to the addition of the DC to the cultures, for the duration of the assay.

FIG. 6 is a bat graph depicting cytokine (IFN-γ) production by MUC-1 specific T cells from immunized MUC-1 transgenic ELISPOT assays carried out using lymph node (LN) cells from immunized and control mice stimulated for 40 hours with 100-mer MUC-1 peptide-pulsed or no peptide control DC.

FIG. 7 is a survival plot showing tumor rejection, plotted as percent surviving at the indicated number of days following tumor challenge for mice treated with MUC-1 peptide microspheres (solid squares) or control/placebo microspheres (solid triangles).

### DETAILED DESCRIPTION OF THE INVENTION

The invention provides a mucin peptide, such as a MUC-1 peptide, encapsulated in a biodegradable polymeric microsphere. The invention also provides an encapsulated nucleic acid encoding a mucin peptide. Surprisingly, the encapsulated mucin peptide or mucin-encoding nucleic acid elicits a stronger immune response and provides improved protection against tumor challenge than direct administration of peptide, alone or with an adjuvant. The compositions of the invention therefore overcome tolerance of helper T cells. The encapsulated mucin peptide or nucleic acid encoding a mucin peptide can be used in a vaccine composition, and can be used in a method for delivering a mucin peptide to a subject, as well as in a method of stimulating an immune response to MUC-1 in a subject, a method of inhibiting tumor growth in a subject having a cancer associated with reduced glycosylation of MUC-1, and in a method of prolonging survival in a subject having a cancer associated with reduced glycosylation of MUC-1.

### Definitions

All scientific and technical terms used in this application have meanings commonly used in the art unless otherwise specified. As used in this application, the following words or phrases have the meanings specified.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “polypeptide” includes proteins, fragments of proteins, and peptides, whether isolated from natural sources, produced by recombinant techniques or chemically synthesized. Polypeptides of the invention typically comprise at least about 8 amino acids.

As used herein, an “immune response” is evidenced by conventional indicators of a protective immune response, including, but not limited to, release of gamma interferon (IFN-γ), T cell proliferation, and cytokine or antibody production.

As used herein, “subject” refers to the recipient of the therapy to be practiced according to the invention. The subject can be any vertebrate, but will preferably be a mammal. If a mammal, the subject will preferably be a human, but may also be a domestic livestock, laboratory subject or pet animal.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, to “prevent” a disease or condition means to hinder or delay the onset or progression of the disease or condition. Prevention includes prophylactic administration of a therapeutic agent that reduces the likelihood or severity of the disease or condition.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, Chapter 43, 14th Ed., Mack Publishing Co, Easton Pa. 18042, USA).

As used herein, “adjuvant” includes those adjuvants, including saponins, commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); QuilA; GPI-100 (Galenica); MPL™ immunostimulant or 3d-MPL (Corixa Corporation); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

### Mucin Peptide and Nucleic Acid Delivery Systems

The invention provides a mucin peptide delivery system comprising one or more mucin peptides encapsulated in biodegradable microspheres. Preferably, the mucin peptides include MUC-1. A particularly preferred MUC-1 peptide comprises at least two tandem repeats of the 20mer sequence, GVTSAPDTRPAPGSTAPPAH (SEQ ID NO:1), and may include 2, 3, 4, 5, 6, 7 or more tandem repeats of the 20 mer sequence. The peptide can be natural or synthetic. Synthetic mucin peptides and their preparation are described in U.S. Pat. Nos. 5,744,144 and 5,829,666, the entire contents of which are incorporated herein by reference. The invention also provides a nucleic acid delivery system comprising one or more nucleic acid molecules encoding one or more mucin peptides, wherein the nucleic acid molecules are encapsulated in biodegradable microspheres.

### Variants

A mucin peptide “variant,” as used herein, is a peptide (or polypeptide) that differs from a native mucin peptide in one or more substitutions, deletions, additions and/or insertions, such that the biological activity of the peptide is not substantially diminished. In the context of the mucin peptides of the invention, biological activity refers to the ability to elicit a specific immune response, as can be assayed using one of the assays described in the examples disclosed herein (e.g., induction of gamma interferon, protection against tumor challenge). In other words, the ability of a variant to specifically bind antibody may be enhanced or unchanged, relative to the native peptide, or may be diminished by less than 50%, and preferably less than 20%, relative to the native peptide. Peptide variants preferably exhibit at least about 80%, more preferably at least about 90% and most preferably at least about 95% identity to the referenced peptides.

Amino acid sequence variants of the peptides are prepared by introducing appropriate nucleotide changes into the encoding DNA, or by peptide synthesis. Such variants include, for example, deletions from, and/or insertions into and/or substitutions of, residues within the amino acid sequence of SEQ ID NO:1 described herein, or variants of other known mucin peptide amino acid sequences. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the peptide that are preferred locations for mutagenesis is called “alanine scanning mutagenesis,” and is described by Cunningham and Wells, 1989, Science, 244:1081-1085. A residue or group of target residues is identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed variants are screened for the desired activity.

Substitution variants have at least one amino acid residue in the molecule removed and a different residue inserted in its place. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1
Conservative Substitutions

| Original Residue | Preferred Substitutions | Exemplary Substitutions |
|---|---|---|
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Hydrophobic: Norleucine, Met, Ala, Val, Leu, Ile;

(2) Neutral hydrophilic: Cys, Set, Thr;

(3) Acidic: Asp, Glu;

(4) Basic: Asn, Gln, His, Lys, Arg;

(5) Residues that influence chain orientation: Gly, Pro; and

(6) Aromatic: Tip, Tyr, Phe.

Non-conservative substitutions are made by exchanging a member of one of these classes for another class.

### Microsphere Formulation

The microspheres of the invention preferably comprise a biodegradable polymer, such as poly(lacto-co-glycolide) (PLG), poly(lactide), poly(caprolactone), poly(hydroxybutyrate) and/or copolymers thereof. Alternatively, the microspheres can comprise another wall forming material. Suitable wall-forming materials include, but ate not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. The delivery system can further comprise an adjuvant, preferably an aminoalkyl glucosaminide 4-phosphate (AGP), 2-deoxy-2-amino-beta-D-glucopyranose (glucosamine) glycosidically linked to a cyclic aminoalkyl (aglycon) group (cyclic AGP), MPL, and/or a saponin such as, for example, QuilA, QS-21 and GPI-100.

In one embodiment, at least about 90% of the microspheres are about 1 to about 20 μm in diameter, more preferably about 3 to about 10 μm, and most preferably, about 6 to about 8 μm in diameter. Microspheres in this size range are well-suited to be phagocytosed by antigen-presenting cells, leading to effective T cell stimulation.

The invention provides a method for encapsulating mucin peptides or nucleic acid molecules in microspheres. The method comprises dissolving a polymer in a solvent to form a polymer solution; adding an aqueous solution containing mucin peptides to the polymer solution to form a primary emulsion; homogenizing the primary emulsion; mixing the primary emulsion with a process medium comprising a stabilizer to form a secondary emulsion; and extracting the solvent from the secondary emulsion to form microspheres encapsulating mucin peptides. For encapsulation of nucleic acid molecules, these method steps are preferably carried out on ice, maintaining a temperature that is above freezing and below 37° C. In one embodiment, the solutions and media are maintained at about 2° C. to about 35° C. In another embodiment, the solutions and media are maintained at about 4° C. to about 25° C. Keeping the materials below 37° C. during the primary and secondary emulsion stages of microsphere preparation can reduce nicking of the DNA. Preserving more of the DNA in a supercoiled form facilitates more efficient transfection of cells. The method can further comprise subsequent steps of washing, freezing and lyophilizing the microspheres.

In a preferred embodiment, the polymer comprises PLG. In some embodiments, the PLG can include ester end groups or carboxylic acid end groups, and have a molecular weight of from about 4 kDa to about 120 kDa, or preferably, about 8 kDa to about 65 kDa. The solvent can comprise, for example, dichloromethane, chloroform, or ethylacetate. In some embodiments, the polymer solution further comprises a cationic lipid and/or an adjuvant, such as MPL. Examples of stabilizers include, but are not limited to, carboxymethylcellulose (CMC), polyvinyl alcohol (PVA), polyvinyl pyrrolidone (PVP), or a mixture thereof. The stabilizer can optionally further comprise a cationic lipid. In some embodiments, the stabilizer comprises from about 0 to about 10% of the process medium, or preferably, about 1% to about 5% of the process medium. In some embodiments, the solvent comprises an internal water volume of from about 0.001% to about 0.5%; and/or the aqueous solution comprises an ethanol content of from about 0% to about 75% (v/v).

In a preferred embodiment, the polymer comprises PLG (RG502H), polyvinyl alcohol is used as a stabilizer, and dichloromethane is used as a solvent. Encapsulation efficiency can be increased with increasing PLG concentration in the organic phase (dichloromethane), in the range of 30-200 mg/ml. These parameters also correlate with an increase in median microsphere diameter (about 1 to about 10 μm).

The selection of the polymer and microsphere formulation can be varied, but is preferably selected to achieve the desired biological activity. In the context of the present invention, the desired biological activity is the ability to effectively deliver a mucin peptide such that, upon administration to the subject, an immune response to MUC-1 is elicited. Preferably, this immune response is sufficient to break tolerance of helper T cells.

The nucleic acid molecule preferably comprises DNA. In one embodiment, the aqueous solution comprises about 0.2 to about 12 mg/ml DNA. The aqueous solution can optionally further comprise a stabilizer, such as BSA, HSA, or a sugar, or an adjuvant, such as an AGP and/or a saponin such as, e.g., QS-21. In one embodiment, the DNA comprises a plasmid of about 2 kb to about 12 kb, preferably, about 3 kb to about 9 kb.

Preferably, at least 50% of the DNA retains a supercoiled formation through the extraction step, more preferably through any subsequent steps, such as lyophilization. Also preferred is a method wherein the encapsulation efficiency is at least about 40%, and/or wherein the microspheres release at least about 50% of the nucleic acid molecules within about 7 days of contact with the desired delivery environment, such as an aqueous environment at 37° C. In a more preferred embodiment, the microspheres release at least about 50% of the nucleic acid molecules within about 4 days.

Because water-soluble agents, such as nucleic acid molecules, do not diffuse through hydrophobic wall-forming materials such as the lactide/glycolide copolymers, pores must be created in the microsphere membrane to allow these agents to diffuse out for controlled-release applications. Several factors will affect the porosity obtained. The amount of agent that is encapsulated affects the porosity of microspheres. Obviously, higher-loaded microspheres (i.e., greater than about 20 wt. %, and preferably between 20 wt. % and 80 wt. %) will be more porous than microspheres containing smaller amounts of agent (i.e., less than about 20 wt. %) because more regions of drug are present throughout the microspheres. The ratio of agent to wall-forming material that can be incorporated into the microspheres can be as low as 0.1% to as high as 80%.

The solvent used to dissolve the wall-forming material will also affect the porosity of the membrane. Microspheres prepared from a solvent such as ethyl acetate will be more porous than microspheres prepared from chloroform. This is due to the higher solubility of water in ethyl acetate than in chloroform. More specifically, during the emulsion step, no solvent is removed from the microdroplets because the process medium is saturated with solvent. Water, however, can dissolve in the solvent of the microdroplets during the emulsion step of the process. By selecting the appropriate solvent or cosolvents, the amount of continuous process medium that will dissolve in the microdroplets can be controlled, which will affect the final porosity of the membrane and the internal structure of the microspheres.

Another factor that will affect the porosity of the membrane is the initial concentration of the wall material/excipient in the solvent. High concentrations of wall material in the solvent result in less porous membranes than do low-concentrations of wall material/excipient. Also, high concentrations of wall material/excipient in the solvent improve the encapsulation efficiency of water-soluble compounds because the viscosity of the solution is higher. Generally, the concentration of wall-forming material/excipient in the solvent will range from about 3% to about 40%, depending on the physical/chemical properties of the wall material/excipient such as the molecular weight of the wall-forming material and the solvent used.

### Compositions

The invention provides compositions that are useful for delivering mucin peptides. In one embodiment, the composition is a pharmaceutical composition. The composition can comprise a therapeutically or prophylactically effective amount of a polynucleotide, recombinant virus, APC or immune cell that encodes or presents one or more mucin peptides, such as the MUC-1 peptide, GVTSAPDTRPAPGSTAPPAH (SEQ ID NO:1), or at least two tandem repeats thereof. Preferably the MUC-1 peptide is about 40, 60, 80, or 105 amino acids in length and comprises 2, 3, 4 or 5 tandem repeats of GVTSAPDTRPAPGSTAPPAH (SEQ ID NO:1). Suitable mucin peptides and methods for preparing them are described in U.S. Pat. Nos. 5,744,144 and 5,827,666, the contents of which are incorporated by reference herein. An effective amount is an amount sufficient to elicit or augment an immune response, e.g., by activating T cells. One measure of the activation of T cells is a cytotoxicity assay or an interferon-gamma release assay, as described in the examples below. In some embodiments, the composition is a vaccine.

In some embodiments, the condition to be treated or prevented is cancer or a precancerous condition (e.g., hyperplasia, metaplasia, dysplasia). Particularly relevant are adenocarcinomas or any cancer associated with reduced glycosylation of O-linked carbohydrates.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. A preferred adjuvant is an AGP, cyclic AGP or MPL. Preferred saponins may be selected from the group consisting of QuilA, QS-21, and GPI-100.

Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive.

Biodegradable microspheres (e.g., polylactate polyglycolate) for use as carriers are disclosed, for example, in U.S. Pat. Nos. 4,897,268; 5,075,109; 5,928,647; 5,811,128; 5,820,883; 5,853,763; 5,814,344; 5,407,609; and 5,942,252; the disclosures of each of which are incorporated herein by reference. In particular, these patents, such as U.S. Pat. No. 4,897,268 and 5,407,609, describe the production of biodegradable microspheres for a variety of uses, but do not teach the optimization of microsphere formulation and characteristics for DNA delivery.

Such compositions may also comprise buffets (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

### Adjuvants

The invention further provides adjuvants for use with vaccines, particularly for use with peptide or DNA vaccines encapsulated in biodegradable microspheres. Such adjuvants comprise an aminoalkyl glucosaminide 4-phosphate (AGP), such as those described in pending U.S. Pat. Nos. 6,113,918 and 6,303,347 and in U.S. patent application Nos. 09/074,720 and 09/905,160, each of which is incorporated herein by reference in its entirety. Another adjuvant preferred for use with the compositions of the invention is 2-deoxy-2-amino-beta-D-glucopyranose (glucosamine) glycosidically linked to a cyclic aminoalkyl (aglycon) group (referred to herein as “cyclic AGP”), as described in U.S. patent application No. 60/223,056.

Compositions of the invention can include an AGP adjuvant and/or additional adjuvants. Most adjuvants contain a substance designed to protect the antigen from rapid catabolism, such as aluminum hydroxide or mineral oil, and a stimulator of immune responses, such as lipid A, Bortadella pertusis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; and monophosphoryl lipid A. Cytokines, such as GM CSF or interleukin-2, -7, or -12, may also be used as adjuvants as may one or more of the saponins such as, for example, QuilA, QS-21, and GPI-100.

Within the vaccines provided herein, the adjuvant composition is preferably designed to induce an immune response predominantly of the Thl type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Hamilton, Mont.) (see US Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555.

Another preferred adjuvant is a saponin, preferably QS-21, which may be used alone or in combination with other adjuvants. QS-21 is a natural saponide molecule purified from the bark of the South American tree, quillaja saponaria Molina. The immunostimulant property of the crude bark extract resides in the saponin fraction. For example, an enhanced system involves the combination of a monophosphoryl lipid A and saponin derivative, such as the combination of QS-21 and 3D-MPL as described in WO 94/00153, or a less reactogenic composition where the QS-21 is quenched with cholesterol, as described in WO 96/33739. MPL comprises a chemically detoxified form of the parent lipopolysaccharide (LPS) from the gram negative bacterium Salmonella minnesota.

Other preferred formulations comprise an oil-in-water emulsion and tocopherol. A particularly potent adjuvant formulation involving QS-21, 3D-MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

### Methods

The invention provides a method for delivering a mucin peptide to a subject. The method comprises administering to the subject a mucin peptide or nucleic acid delivery system, or a composition, of the invention. The invention further provides a method of stimulating an immune response to MUC-1 in a subject, a method of inhibiting tumor growth in a subject having a cancer associated with reduced glycosylation of MUC-1, a method of prolonging survival in a subject having a cancer associated with reduced glycosylation of MUC-1, as well as a method for treating or preventing a cancer associated with reduced glycosylation of MUC-1. The method comprises administering to the subject a composition or delivery system comprising a MUC-1 peptide or nucleic acid of the invention. Administration can be performed as described herein.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases, the physician needs to evaluate the production of an immune response against the pathogen, progression of the disease, and any treatment-related toxicity.

Administration by many of the routes of administration described herein or otherwise known in the art may be accomplished simply by direct administration using a needle, catheter or related device, at a single time point or at multiple time points.

### EXAMPLES

The following examples are presented to illustrate the present invention and to assist one of ordinary skill in making and using the same. The examples are not intended in any way to otherwise limit the scope of the invention.

### Example 1

### MUC-1 Peptide Antigens

MUC-1 peptides employed in the formulations, compositions and methods of the present invention comprises two or more repeats of the 20-mer sequence GVTSAPDTRPAPGST APPAH (SEQ ID NO:1) from the extracellular tandem repeat domain of MUC-1. Peptides were synthesized as described in Soares et al., J. Immuno 166(11):6555-63 (2001) using a Chemtech 200 machine with N-(9-fluorenyl)methoxycarbonyl chemistry and purified by HPLC. As indicated in further detail below, peptides employed in the presently disclosed examples comprised two or five tandem repeats of the 20-mer sequence to create 40-mer and 100-mer MUC-1 peptides, respectively.

### Example 2

### Vaccines and Immunization Protocols

Three different immunization protocols were tested in vivo. Mice (4-6 week-old MUC-1 C57BL/6 or MUC-1 transgenic mice on C57BL/6 background) were immunized with: 1) synthetic MUC-1 peptide (100 μg/mouse) coadministered with soluble murine GM-CSF (2 μg/mouse; Immunex Corp., Seattle, Wash.) injected s.c.; 2) synthetic MUC1 peptide (100 μg/mouse) coadministered with SP-AS2 (50 μg/mouse; SmithKline Beecham Biologicals, Rixensatt, Belgium) injected i.m.; or 3) murine DC prepulsed with 20 μg/ml of synthetic MUC-1 peptide in AIM-V medium (Life Technologies, Grand Island N.Y.) overnight (2-5×10⁴ DC/mouse injected s.c.). SB-AS2 is an oil-in water emulsion containing 3-deacylated-monophosphoryl lipid A, a detoxified form of lipid A, and purified fraction number 21 of Quillaria saponaria, known as QuilA. The DC were generated as described in Mayordomo, et al., Nature Med. 1:1297 (1995), the major modification being that they were grown in serum-free medium. Briefly, they were differentiated in vitro from bone marrow precursors with murine GM-CSF (10 ng/ml) and murine IL-4 (10 ng/ml) in AIM-V medium for 7 days. On day 7, the DC were purified on Nycoprop gradient (Nycomed, Oslo, Norway), pulsed overnight with peptide in Teflon vials, and washed before vaccination. For the DC vaccine containing soluble peptide, soluble MUC-1 peptide was added to the washed peptide-pulsed DC at a final concentration of 100 μ/mouse before vaccination. The mice were immunized once and boosted twice at 3-wk intervals in the right hind flank.

### Example 3

### The MUC-1 Peptide Induces IFN-γ as Measured by Enzyme-linked Immunospot (EISPOT) Assay

Lymph node (LN) cells were mixed with peptide-pulsed bone marrow derived DC (at a ratio of 10:1) in MultiScreen 96-well filtration plates (Millipore, Bedford, Mass.) precoated with the anti-IFN-γ capture Ab (BD Pharmingen, San Jose, Calif.). The plates were incubated for 40 h at 37° C. After three washes with PBS/0.1% Tween 20, the plates were incubated with 2 μg/well of biotin-labeled anti-IFN-γ Ab (BD Pharmingen) at 37° C. The plates were washed, and spots developed with the Elite Vectastain ABC Kit (Vector Laboratories, Burlingame, Calif.). For blocking studies, anti-CD4, anti-CD8, or isotype control Abs (BD Pharmingen) were added to the wells at a final concentration of 2.5 μg/ml.

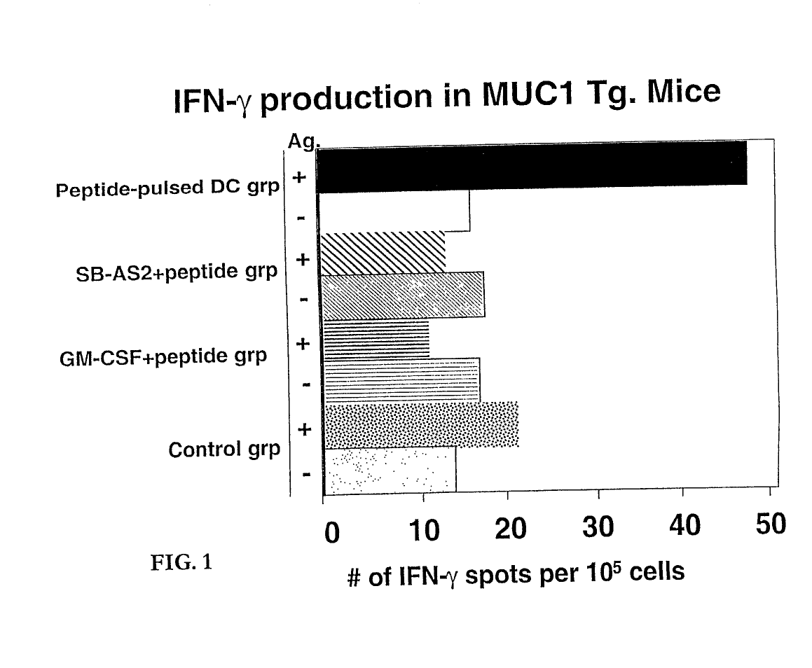

FIG. 1 is a bar graph showing interferon gamma (IFN-γ) production in MUC-1 transgenic mice treated with MUC-1 peptide, as measured by number of IFN-γ spots per 10⁵ cells in peptide-pulsed dendritic cell group (solid and white bars), AS2+ peptide group (diagonally striped bars), GM-CSF+ peptide group (striped bars), and control group (stippled bars); + indicates antigen-positive and − indicates antigen-negative mice.

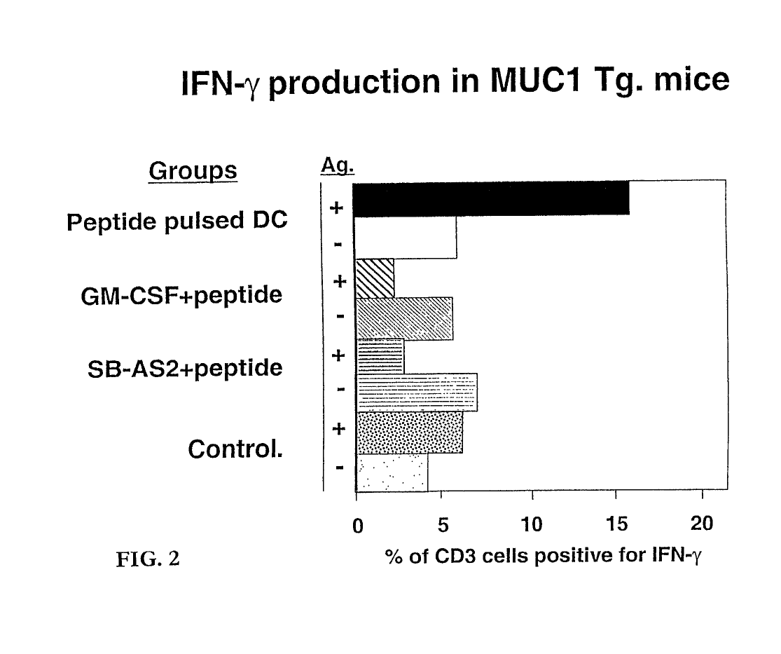

FIG. 2 is a bar graph showing interferon gamma (IFN-γ) production in mice treated with MUC-1 peptide, as measured by percentage of CD3 cells positive for IFN-γ in peptide-pulsed dendritic cell group (solid and white bars), AS2+ peptide group (diagonally striped bars), GM-CSF+ peptide group (striped bars), and control group (stippled bars);+ indicates antigen-positive and − indicates antigen-negative mice.

### Example 4

### Mucin Peptides Protect Mice From Tumor Challenge

The T cell lymphoma MUC-1 transfectant RMA-MUC-1 on a C57BL/6 background expresses both the fully glycosylated and underglycosylated forms of MUC-1. Ten days following the last boost, the mice were anesthetized with Metofane (Schering-Plough Animal Health, Omaha, Nebr.) and 5×10⁴ RMA-MUC-1 cells injected subcutaneously in the shaved right hind flank. Tumor growth was monitored every 2-3 days and tumor size determined using calipers. Mice were sacrificed when the tumor size reached 2 cm in diameter.

In this example, mucin peptides were delivered either as pulsed DC, in combination with GM-CSF or in combination with SB-AS2 to tumor-challenged wild-type or MUC-1 transgenic (tg) mice and their efficacy was compared to that of a negative control group not receiving the Muc-1 peptide. The MUC-1 peptide used was a 40mer comprising two repeats of the 20 mer sequence: GVTSAPDTRPAPGST APPAH (SEQ ID NO:1).

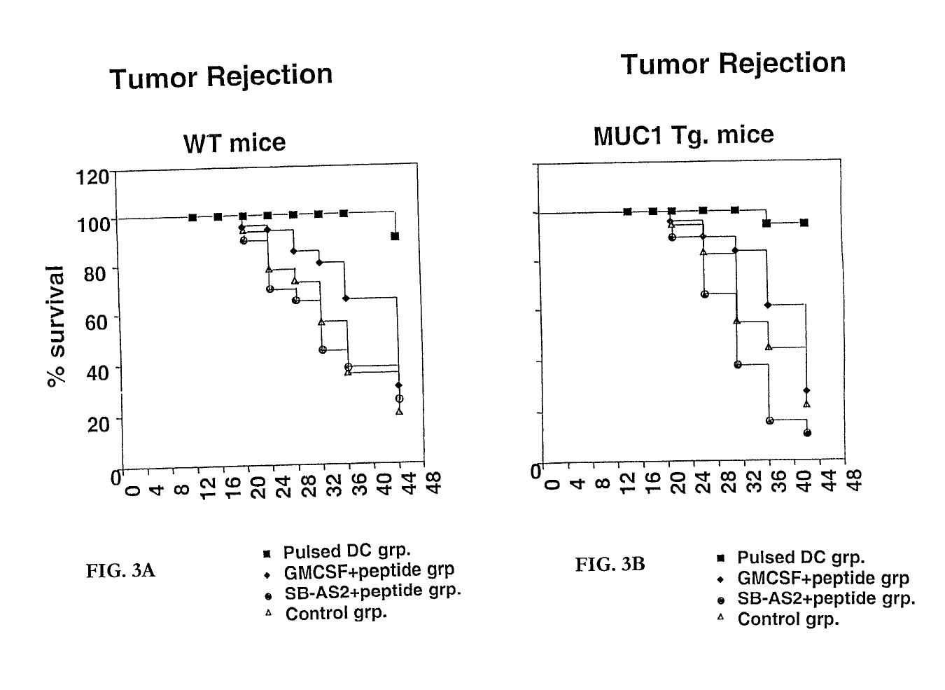

FIG. 3 is a survival plot showing tumor rejection, plotted as percent surviving at the indicated number of days following tumor challenge for wild type mice (left panel) and MUC-1 transgenic (right panel) mice. Groups were treated as follows: dendritic cells pulsed with MUC-1 peptide (squares), GM-CSF+ peptide (diamonds), AS2+ peptide (circles), and control (triangles).

### Example 5

### Encapsulation of Mucin Peptides in PLG Microspheres

This example describes the formulation of poly(lactide-co-glycolide) (PLG) microspheres suitable for encapsulating and delivering mucin peptides. The microspheres were prepared using a double emulsion technique (J. H. Eldridge et al. Mol Immunol, 28:287-294, 1991; S. Cohen et al. Pharm Res, 8:713-720, 1991). RG502H was used as the polymer, and polyvinyl alcohol was used as a stabilizer. Encapsulation efficiency was found to increase with increasing PLG concentration in the organic phase (dichloromethane) (30-200 mg/ml), which also correlated with an increase in median microsphere diameter (about 1 to about 10 μm).

### Example 6

### Mucin Peptides Encapsulated in PLG Microspheres do not Elicit MUC-1 Specific Antibodies

In this example, mucin peptides were delivered in microspheres to tumor-challenged mice and their efficacy was compared to that of placebo-microspheres. The MUC-1 peptide used was a 40 mer comprising two repeats of the 20 mer sequence: GVTSAPDTRPAPGST APPAH (SEQ ID NO:1). PLG microspheres (mean diameter 7 μ) were loaded internally with 0.81% w/w of peptide. Mice were immunized with 10 μg of peptide equivalents of MUC-1 PLG microspheres/100 μl PBS or an equivalent weight of placebo microspheres resuspended in LPS-free PBS. The mice were immunized once and boosted twice, at three-week intervals.

Ten days following the last boost, blood samples were collected by tail bleeding and the serum tested for the presence of MUC-1 specific antibodies using a MUC-1 specific ELISA assay. Kotera et al., Cancer Res. 54(11):2856-60 (1994). 96-well Immulon 4 plates (Dynatech, Chantilly, Va.) were coated at room temperature overnight with 10 μg/ml of 100 amino acid long MUC-1 peptide (five tandem repeats of 20-mer sequence) in phosphate buffered saline. The plates were washed three times with PBS and incubated with serial dilutions of the immune serum for 1 hour at room temperature. Following three washes with PBS/0.1% Tween 20, the plates were incubated with goat anti-mouse peroxidase-conjugated secondary antibodies for 1 hour at room temperature. The goat anti-mouse-IgM and -IgG secondary antibodies were obtained from Sigma (St. Louis, Mo.). The goat anti-mouse-IgG1, -IgG2b and -IgG3 antibodies were obtained from Southern Biotechnology Associates, Inc. (Birmingham, Ala.). The plates were washed three times with PBS/0.1% Tween-20 and then incubated with the substrate O-phenylenediamine dihydrochloride tablets (Sigma, St. Louis, Mo.) for 1 hour. The reaction was stopped using 2.5 M sulfuric acid and the absorbance measured at 490 nm.

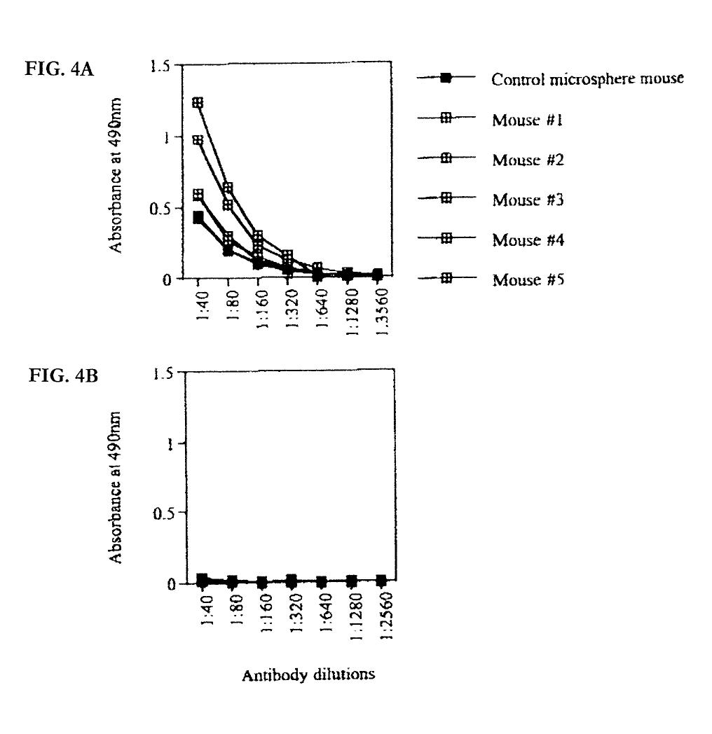

The data shown in FIG. 4 reveal that no MUC-1 specific IgM or IgG response was elicited by the MUC-1 PLG microsphere vaccine. Without being limited to any particular theory or operation, these data suggest the possibility that the PLG microspheres of the present Example do not release peptide unless they are degraded. Thus, it appears that the amount of free soluble peptide released from the degraded microspheres is insufficient to activate B cells to produce MUC-1 specific antibodies and further suggests that MUC-1 specific cellular immunity is necessary and sufficient for tumor immunity.

### Example 7

### MUC-1 Peptides Encapsulated in PLG Microspheres Elicit MUC-1 Specific T Cells in MUC-1 Transgenic Mice

Lymph node cells were mixed with peptide-pulsed bone marrow-derived DC (at a ratio of 10:1) in MultiScreen 96-well filtration plates (Millipore, Bedford, Mass.) precoated with the anti-IFN-γ capture antibody (Pharmingen, San Jose, Calif.). The plates were incubated for 40 hours at 37° C. Following three washes with PBS/0.1% Tween20, the plates were incubated with 2 μ/well of biotin labeled anti-IFN-γ antibody (Pharmingen, San Jose, Calif.) at 37° C. The plates were washed, and spots developed using the Elite Vectastain ABC Kit (Vector Laboratories Inc., Burlingame, Calif.). For blocking studies, anti-CD4, anti-CD8, or isotype control antibodies (Pharmingen, San Jose, Calif.) were added to the wells at a final concentration of 2.5 μg/ml.

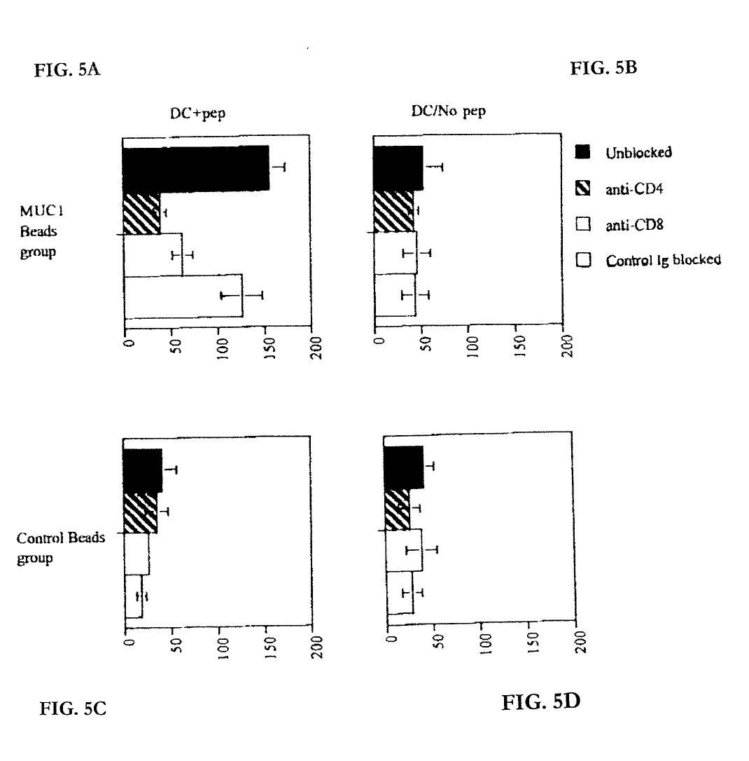

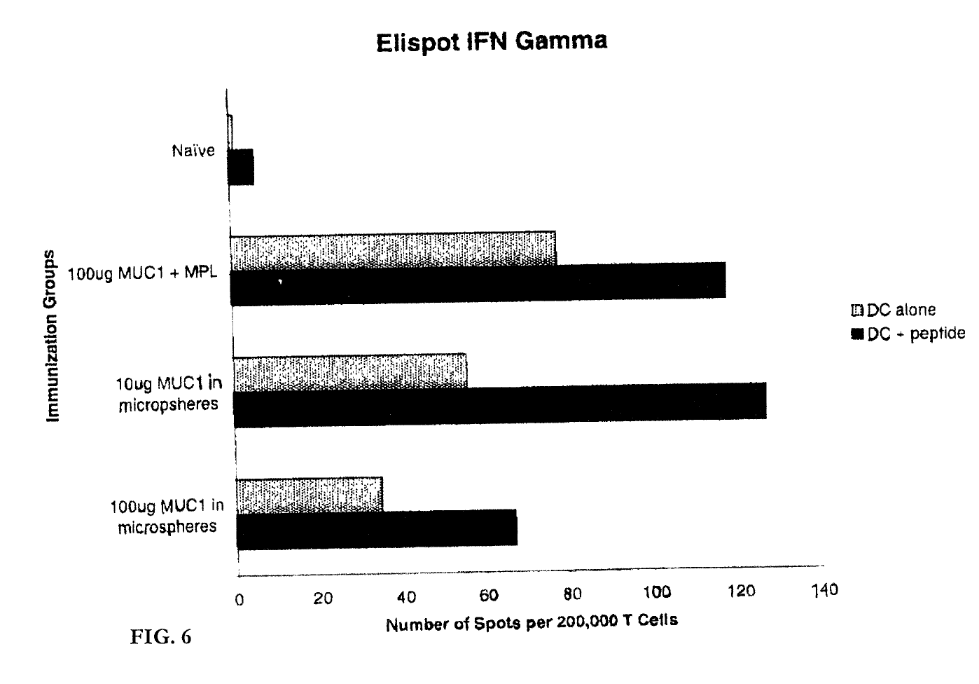

As shown in FIGS. 5 and 6, 40-mer and 100-mer MUC-1 loaded microspheres, respectively, were both capable of inducing IFN-γ producing T cells in MUC-1 transgenic mice. The significantly low number of background spots obtained in the ELISPOT assay using DC alone, as stimulators, suggests that these T cells ate MUC-1 specific. Moreover, blocking with anti-CD4 or anti-CDS antibodies resulted in a significant decrease in the total number of IFN γ spots indicating that the vaccine activated both CD4+ and CD8+ cells. In addition, despite the induction of MUC-1 specific CD4+ T cells, and as disclosed in Example 6, no MUC-1 specific IgG were induced following immunization.

### Example 8

### Mucin Peptides Encapsulated in PLG Microspheres Protect Mice From Tumor Challenge

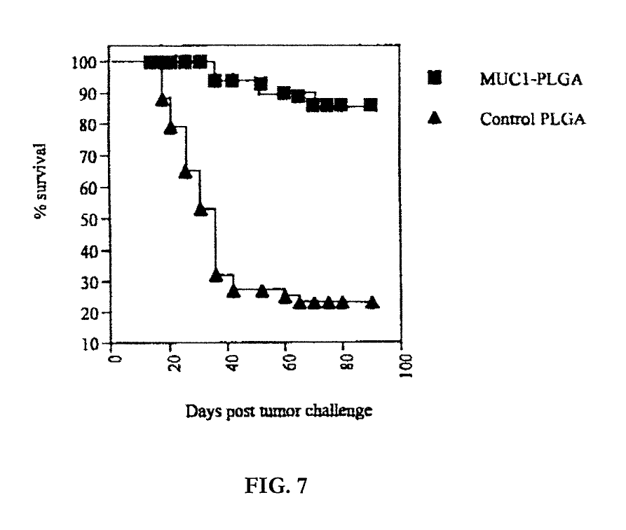

Mice were challenged 10 days after the last immunization with RMA-MUC-1 tumor cells injected subcutaneously on the right hind flank, and tumor growth was monitored up to 90 days. By day 35, approximately 70% of MUC-1 transgenic mice immunized with control unloaded microspheres were sacrificed because their tumors reached 2 cm. As seen in FIG. 7, the 40-mer MUC-1 peptide-microspheres exhibited significantly better protection from tumor challenge than placebo-microspheres, as measured by survival time after tumor challenge. 86% of immunized mice survived, tumor free, up to 90 days post-tumor challenge.

### Example 9

### MUC-1 Transgenic Mice Immunized with MUC-1 PLG Microspheres Display no Signs of Autoimmunity in MUC-1 Expressing Tissues Following Immunization or Tumor Rejection

To investigate whether the immune responses elicited by vaccination alone or those further boosted through tumor rejection, would show reactivity against normal tissues, MUC-1 expressing tissues, MUC-1 expressing lung, pancreas, liver and kidney were harvested from MUC-1 transgenic mice following immunization and post tumor rejection. Mononuclear cellular infiltrates into these tissues, especially around the MUC-1+ ducts or tissue destruction in H&E stained tissue sections, were considered to be signs of autoimmunity.

There were no obvious mononuclear infiltrates into the pancreas or lung in MUC-1 transgenic mice, when the tissues were harvested from immunized mice 10 days following the last boost. There were also no signs of tissue destruction of these MUC-1 expressing tissues. In the kidney, however, large cellular infiltrates were observed in kidneys from MUC-1 PLGA immunized and control MUC-1 transgenic mice suggesting that kidney infiltrates were not the consequence of immunization.

Observation of stained tissues harvested from mice on days 60 and 90 post-tumor challenge revealed no tissue infiltration into or destruction of the pancreas and lung. As before, cellular infiltrates in the kidneys of mice that received MUC-1 PLG or control microspheres was observed suggesting that the infiltration of the kidney was independent of the immunization and tumor rejection. Accordingly, these data demonstrate that immunization with MUC-1 peptide-loaded PLG microspheres induced tumor rejection responses that did not result in damage of normal MUC-1+ tissues.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A composition comprising a mucin peptide and a biodegradable polymeric microsphere.

2. The composition of claim 1, wherein the mucin peptide comprises a MUC-1 peptide.

3. The composition of claim 2, wherein the MUC-1 peptide comprises at least two tandem repeats of the 20mer sequence, GVTSAPDTRPAPGSTAPPAH (SEQ ID NO: 1).

4. The composition of claim 3, wherein the MUC-1 peptide comprises 2, 3, 4, 5, 6 or 7 tandem repeats of the 20 mer sequence, GVTSAPDTRPAPGSTAPPAH (SEQ ID NO: 1).

5. The composition of claim 1, wherein the microsphere comprises poly(lacto-co-glycolide) (PLG), poly(lactide), poly(caprolactone), poly(hydroxybutyrate) and/or a copolymer thereof.

6. The composition of claim 5, wherein the microsphere comprises poly(lacto-co-glycolide) (PLG).

7. The composition of claim 1, further comprising an adjuvant and/or a saponin.

8. The composition of claim 7, wherein the adjuvant is selected from the group consisting of MPL, an aminoalkyl glucosaminide 4-phosphate (AGP), and 2-deoxy-2-amino-beta-D-glucopyranose (glucosamine) glycosidically linked to a cyclic aminoalkyl (aglycon) group (cyclic AGP); and the saponin is selected from the group consisting of QuilA, QS-21 and GPI-100.

9. The composition of claim 1, which comprises a plurality of microspheres and wherein at least about 90% of the microspheres are from about 1 μm to about 20 μm.

10. The composition of claim 9, wherein at least about 90% of the microspheres are from about 3 μm to about 10 μm.

11. The composition of claim 10, wherein at least about 90% of the microspheres are from about 6 μm to about 8 μm.

12. A method for encapsulating mucin peptides in microspheres comprising:
(a) dissolving a polymer in a solvent to form a polymer solution;
(b) adding an aqueous solution containing mucin peptides to the polymer solution to form a primary emulsion;
(c) homogenizing the primary emulsion;
(d) mixing the primary emulsion with a process medium comprising a stabilizer to form a secondary emulsion; and
(e) extracting the solvent from the secondary emulsion to form microspheres encapsulating mucin peptides.

13. The method of claim 12, wherein the polymer comprises poly(lacto-co-glycolide) (PLG), poly(lactide), poly(caprolactone), poly(hydroxybutyrate) and/or a copolymer thereof.

14. The method of claim 13, wherein the PLG has a molecular weight of from about 8 kDa to about 65 kDa.

15. The method of claim 12, wherein the polymer solution further comprises an adjuvant and/or a saponin.

16. The method of claim 15, wherein the adjuvant comprises MPL, AGP or cyclic AGP; and the saponin comprises QuilA, QS-21 or GPI-100.

17. The method of claim 12, wherein the mucin peptide comprises MUC-1.

18. The method of claim 12, wherein at least about 90% of the microspheres are from about 1 μm to about 20 μm.

19. The method of claim 18, wherein at least about 90% of the microspheres are from about 3 μm to about 10 μm.

20. The method of claim 19, wherein at least about 90% of the microspheres are from about 6 μm to about 8 μm.

21. An encapsulated mucin peptide produced by the method of claim 12.

22. A vaccine comprising the composition of claim 1 or the mucin peptide of claim 21 and a pharmaceutically acceptable carrier.

23. The vaccine of claim 22, further comprising an adjuvant and/or a saponin.

24. The vaccine of claim 23, wherein the adjuvant comprises MPL, AGP or cyclic AGP; and the saponin comprises QuilA, QS-21 or GPI-100.

25. A method for delivering a mucin peptide to a subject comprising administering to the subject a vaccine of claim 22.

26. A method of stimulating an immune response to MUC-1 in a subject comprising administering a vaccine of claim 22 to the subject.

27. A method of inhibiting tumor growth in a subject having a cancer associated with reduced glycosylation of MUC-1 comprising administering a vaccine of claim 22 to the subject.

28. A method of prolonging survival in a subject having a cancer associated with reduced glycosylation of MUC-1 comprising administering a vaccine of claim 22 to the subject.

29. A method of treating or preventing a cancer associated with reduced glycosylation of MUC-1 comprising administering a vaccine of claim 22 to the subject.


SEQUENCE LISTING (1 sequence)

SEQ ID NO: 1 - protein, 20 aa, Homo sapiens
GVTSAPDTRPAPGSTAPPAH